SEQUENCE LISTING (8 sequences)

SEQ ID NO: 1 - DNA, 66 bp, Artificial Sequence
  TGF-beta antisense oligonucleotides
ctcaccagagcccgcgtgctaatggtggacaaaaaacccgcgtgctaatggtggacaagtcctgtc

SEQ ID NO: 2 - DNA, 79 bp, Artificial Sequence
  scrambled TGF-beta antisense oligonucleotides
ctcaccagagccaccaccaccaacaccaccaccaccaaaaaaaccaccaccaccaacaccaccaccaccaagtcctgtc

SEQ ID NO: 3 - DNA, 20 bp, Artificial Sequence
  RCA primer
ctctggtgaggacaggactt

SEQ ID NO: 4 - protein, 47 aa, Unknown
  hBD23 amino acid
GTQRCWNLYGKCRYRCSKKERVYVYCINNKMCCVKPKYQPKERWWPF

SEQ ID NO: 5 - DNA, 25 bp, Artificial Sequence
  TGF-beta_Forward
cgaagcggactactatgctaaagag

SEQ ID NO: 6 - DNA, 23 bp, Artificial Sequence
  TGF-beta_Reverse
tggttttctcatagatggcgttg

SEQ ID NO: 7 - DNA, 19 bp, Artificial Sequence
  GAPDH_Forward
tgcaccaccaactgcttag

SEQ ID NO: 8 - DNA, 18 bp, Artificial Sequence
  GAPDH_Reverse
ggatgagggatgatgttc

(19) United States
(12) Patent Application Publication
(10) Pub. No.: US 2021/0130831 A1
(43) Pub. Date: May 6, 2021

(54) LUNG-SPECIFIC DRUG DELIVERY SYSTEM CONSISTING OF OLIGONUCLEOTIDE POLYMERS AND BIOCOMPATIBLE CATIONIC PEPTIDES FOR THE PREVENTION OR TREATMENT OF PULMONARY FIBROSIS AND USE THEREOF

(71) Applicant: KOREA INSTITUTE OF SCIENCE AND TECHNOLOGY, Seoul (KR)

(72) Inventors: Dae-Ro AHN, Seoul (KR); Junghyun KIM, Seoul (KR); Seong Jae KANG, Seoul (KR)

(21) Appl. No.: 17/084,906

(22) Filed: Oct. 30, 2020

(30) Foreign Application Priority Data
Nov. 1, 2019 (KR) 10-2019-0138845

(51) Int. Cl. C12N 15/113; A61K 47/64; A61P 11/00

(52) U.S. Cl. CPC C12N 15/1136; A61K 47/6455; C12N 2320/32; C12N 2310/11; C12N 2310/51; A61P 11/00

(57) ABSTRACT

The present invention relates to a lung-specific drug delivery system consisting of oligonucleotide polymers and biocompatible cationic peptides for the prevention or treatment of pulmonary fibrosis, and a use thereof. The drug delivery system according to the present invention can be specifically accumulated in the lungs and absorbed into lung fibrotic cells to knock down TGF-β, thereby preventing or treating pulmonary fibrosis.

### CROSS-REFERENCE TO RELATED APPLICATION

This application claims the benefit of Korean Patent Application No. 10-2019-0138845, filed on Nov. 1, 2019, in the Korean Intellectual Property Office, the disclosure of which is incorporated herein in its entirety by reference.

### SEQUENCE LISTING

A Sequence Listing, incorporated herein by reference, is submitted in electronic form as an ASCII text file, created Oct. 29, 2020, having size 4.0 Kb, and named “8N33568.txt”.

### TECHNICAL FIELD

The present invention relates to a lung-specific drug delivery system consisting of oligonucleotide polymers and biocompatible cationic peptides for the prevention or treatment of pulmonary fibrosis, and a use thereof.

### BACKGROUND ART

Pulmonary fibrosis is a chronic disease based on the excessive production of extracellular matrix, caused by an overexpressed inflammatory activity on environmentally or chemically damaged lung tissue [1,2]. Abnormal accumulation of fibrous tissue narrows the airways and thickens the epithelium in the alveoli, causing a decrease in blood oxygen supply, thereby leading to serious breathing problems [3,4].

The incidence of pulmonary fibrosis is increasing, but the current treatment methods are very limited [5-7]. During the progression of fibrosis, many growth factors are up-regulated [8]. In particular, TGF-β is known to play a central role in the fibro-proliferative process in general, suggesting that TGF-β and its downstream genes can be potential targets for the treatment of the disease [10-12].

Oligonucleotide-based gene modulators, such as antisense oligonucleotides (ASOs) and small interfering RNAs (siRNAs) targeting mRNA, are promising molecules to treat diseases refractory to treatment with small-molecule drugs [13-15]. These can be simply designed based on the target sequence, without consideration of complex protein structures, and provide knockdown with relatively low toxicity to targets [13].

Previously, ASOs and siRNAs targeting TGF-β and the relevant downstream factors have been utilized as potential therapeutic agents for the treatment of fibrosis in other organs, such as the heart, kidneys, and intestines [16-19]. However, as TGF-β plays active roles at other sites [20], a lung-specific delivery using oligonucleotides or the like is necessary to prevent the potential side effects from the undesired suppression of the target in other tissues.

Although lung delivery via local routes can considerably avoid drug distribution to other tissues, several drawbacks need to be addressed. Intratracheal injection is an invasive approach and is not acceptable in practical applications. Intranasal administration, using nose breathers demonstrated in a mouse model, is not applicable into clinical practice owing to differences in lung anatomy [21-23]. While non-invasive inhalation is a widely used method for lung delivery, pulmonary surfactants, such as mucus and alveolar fluid, severely reduce cellular penetration and transfection of the oligonucleotides [24]. Moreover, many lung diseases including fibrosis occur in the lower region of the lung, which is not easily accessible by improperly sized aerosols [25].

These drawbacks of local delivery methods could be addressed by employing a systemic delivery route, such as intravenous injection, since the lower region of the lung is directly accessible through capillary blood vessels surrounding the alveoli, circumventing the interference of pulmonary surfactants. However, systemic delivery of oligonucleotide therapeutics to the fibrotic region of the lung is very challenging, as it requires a delivery platform that can achieve multiple goals, including protection of oligonucleotides against nuclease, lung-specific distribution of the oligonucleotides, and delivery of distributed oligonucleotides into the fibrotic cells.

With this background, in order to develop a delivery platform that achieves the multiple conditions described above and to treat pulmonary fibrosis, the present inventors utilized a biocompatible cationic peptide as a delivery system for systematic lung-specific delivery of an ASO targeting TGF-β mRNA. As a result, the present invention was completed by confirming that ASO successfully delivered to the lungs effectively downregulated the target to suppress pulmonary fibrosis in an animal model.

### PRIOR LITERATURE

### Non-Patent Literature

[1] H. Tanjore, X. C. Xu, V. V. Polosukhin, A. L. Degryse, B. Li, W. Han, T. P. Sherrill, D. Plieth, E. G. Neilson, T. S. Blackwell, W. E. Lawson, Contribution of Epithelial-derived Fibroblasts to Bleomycin-induced Lung Fibrosis, American Journal of Respiratory and Critical Care Medicine 180(7) (2009) 657-665.
[2] Z. Chen, Z. Wu, W. Ning, Advances in Molecular Mechanisms and Treatment of Radiation-Induced Pulmonary Fibrosis, Translational Oncology 12(1) (2019) 162-169.
[3] I. E. Fernandez, O. Eickelberg, New cellular and molecular mechanisms of lung injury and fibrosis in idiopathic pulmonary fibrosis, The Lancet 380(9842) (2012) 680-688.
[4] P. J. Sime, K. M. A. O'Reilly, Fibrosis of the Lung and Other Tissues: New Concepts in Pathogenesis and Treatment, Clinical Immunology 99(3) (2001) 308-319.
[5] J. Hutchinson, A. Fogarty, R. Hubbard, T. McKeever, Global incidence and mortality of idiopathic pulmonary fibrosis: a systematic review, European Respiratory Journal 46(3) (2015) 795-806.
[6] R. J. Mason, M. I. Schwarz, G. W. Hunninghake, R. A. Musson, Pharmacological Therapy for Idiopathic Pulmonary Fibrosis, American Journal of Respiratory and Critical Care Medicine 160(5) (1999) 1771-1777.
[7] I. E. Fernandez, O. Eickelberg, The impact of TGF-β on lung fibrosis: from targeting to biomarkers, Proceedings of the American Thoracic Society 9(3) (2012) 111-116.
[8] T. A. Wynn, T. R. Ramalingam, Mechanisms of fibrosis: therapeutic translation for fibrotic disease, Nature Medicine 18 (2012) 1028.
[9] L. L. McCormick, Y. Zhang, E. Tootell, A. C. Gilliam, Anti-TGF-β Treatment Prevents Skin and Lung Fibrosis in Murine Sclerodermatous Graft-Versus-Host Disease: A Model for Human Scleroderma, The Journal of Immunology 163(10) (1999) 5693-5699.
[10] T. J. Broekelmann, A. H. Limper, T. V. Colby, J. A. McDonald, Transforming growth factor beta 1 is present at sites of extracellular matrix gene expression in human pulmonary fibrosis, Proceedings of the National Academy of Sciences 88(15) (1991) 6642-6646.
[11] S. M. Bentzen, J. Z. Skoczylas, M. Overgaard, J. Overgaard, Radiotherapy-Related Lung Fibrosis Enhanced by Tamoxifen, JNCI: Journal of the National Cancer Institute 88(13) (1996) 918-922.
[12] T. J. Gross, G. W. Hunninghake, Idiopathic Pulmonary Fibrosis, New England Journal of Medicine 345(7) (2001) 517-525.
[13] N. M. Dean, C. F. Bennett, Antisense oligonucleotide-based therapeutics for cancer, Oncogene 22(56) (2003) 9087-9096.
[14] C. A. Stein, D. Castanotto, FDA-Approved Oligonucleotide Therapies in 2017, Molecular Therapy 25(5) (2017) 1069-1075.
[15] Y. Dong, D. J. Siegwart, D. G. Anderson, Strategies, design, and chemistry in siRNA delivery systems, Advanced Drug Delivery Reviews (2019).
[16] E. van Rooij, L. B. Sutherland, J. E. Thatcher, J. M. DiMaio, R. H. Naseem, W S. Marshall, J. A. Hill, E. N. Olson, Dysregulation of microRNAs after myocardial infarction reveals a role of miR-29 in cardiac fibrosis, Proceedings of the National Academy of Sciences 105(35) (2008) 13027-13032.
[17] M. Hwang, H.-J. Kim, H.-J. Noh, Y-C. Chang, Y-M. Chae, K.-H. Kim, J.-P. Jeon, T.-S. Lee, H.-K. Oh, Y.-S. Lee, K.-K. Park, TGF-β1 siRNA suppresses the tubulointerstitial fibrosis in the kidney of ureteral obstruction, Experimental and Molecular Pathology 81(1) (2006) 48-54.
[18] K. Cheng, N. Yang, R. I. Mahato, TGF-β1 Gene Silencing for Treating Liver Fibrosis, Molecular Pharmaceutics 6(3) (2009) 772-779.
[19] R. Izzo, G. Bevivino, V. De Simone, S. Sedda, I. Monteleone, I. Marafini, M. Di Giovangiulio, A. Rizzo, E. Franzè, A. Colantoni, A. Ortenzi, G. Monteleone, Knockdown of Smad7 With a Specific Antisense Oligonucleotide Attenuates Colitis and Colitis-Driven Colonic Fibrosis in Mice, Inflammatory Bowel Diseases 24(6) (2018) 1213-1224.
[20] D. A. Clark, R. Coker, Molecules in focus Transforming growth factor-beta (TGF-β), The International Journal of Biochemistry & Cell Biology 30(3) (1998) 293-298.
[21] D. S. Southam, M. Dolovich, P. M. O'Byrne, M. D. Inman, Distribution of intranasal instillations in mice: effects of volume, time, body position, and anesthesia, American Journal of Physiology-Lung Cellular and Molecular Physiology 282(4) (2002) L833-L839.
[22] C. Egger, C. Cannet, C. Gerard, E. Jarman, G. Jarai, A. Feige, T. Suply, A. Micard, A. Dunbar, B. Tigani, N. Beckmann, Administration of Bleomycin via the Oropharyngeal Aspiration Route Leads to Sustained Lung Fibrosis in Mice and Rats as Quantified by UTE-MRI and Histology, PLOS ONE 8(5) (2013) e63432.
[23] A. J. Hickey, L. Garcia-Contreras, Immunological and Toxicological Implications of Short-Term Studies in Animals of Pharmaceutical Aerosol Delivery to the Lungs: Relevance to Humans, 18(4) (2001) 45.
[24] I. Roy, N. Vij, Nanodelivery in airway diseases: Challenges and therapeutic applications, Nanomedicine: Nanotechnology, Biology and Medicine 6(2) (2010) 237-244.
[25] J. K.-W. Lam, W. Liang, H.-K. Chan, Pulmonary delivery of therapeutic siRNA, Advanced Drug Delivery Reviews 64(1) (2012) 1-15.

### DISCLOSURE

### Technical Problem

An object of the present invention is to provide a lung-specific drug delivery system consisting of oligonucleotide polymers, which include a repeating unit of an antisense against TGF-β, and biocompatible cationic peptides.

Another object of the present invention is to provide a pharmaceutical composition for the prevention or treatment of pulmonary fibrosis, comprising, as an active ingredient, a lung-specific drug delivery system consisting of oligonucleotide polymers, which include a repeating unit of an antisense against TGF-β, and biocompatible cationic peptides.

Still another object of the present invention is to provide a method for preventing or treating pulmonary fibrosis, comprising a step of administering to a subject a composition comprising a lung-specific drug delivery system consisting of oligonucleotide polymers, which include a repeating unit of an antisense against TGF-β, and biocompatible cationic peptides.

### Technical Solution

The present invention will be described in detail as follows. Meanwhile, each description and embodiment disclosed in the present invention can also be applied to each of the other descriptions and embodiments. That is, all combinations of various elements disclosed in the present invention belong to the scope of the present invention. In addition, the scope of the present invention cannot be considered as being limited by the specific description provided below.

One aspect of the present invention for achieving the objects described above provides a lung-specific drug delivery system consisting of oligonucleotide polymers, which include a repeating unit of an antisense against TGF-β, and biocompatible cationic peptides.

In the present invention, the term “oligonucleotide” refers to a synthesized short-stranded DNA or RNA molecule.

In the present invention, the term “oligonucleotide polymer” means that one or more oligonucleotides as monomers are linked to form a polymer.

In the present invention, the term “antisense oligonucleotide (ASO)” refers to a single-stranded DNA or RNA that is complementary to a specific gene sequence, and is used to reduce the level of protein synthesis by inhibiting the processing and translation of mRNA.

When an ASO works normally, the portion where the DNA/RNA double strand is formed between mRNA and an ASO may be degraded by an RNase H enzyme, thereby inhibiting mRNA processing and translation. In addition, gene expression may be reduced using a molecular tool called “Morpholino” that causes blocking of RNA splicing proteins. The antisense oligonucleotides may be used interchangeably with antisenses.

In the present invention, the specific gene sequence described above may be a gene sequence encoding transforming growth factor beta (TGF-β) including TGF-β1, 2, and 3 subfamilies, and specifically may be a gene sequence encoding any one or more selected from the group consisting of TGF-β1, TGF-β2, and TGF-β3, and more specifically may be a gene sequence encoding TGF-β1, but the specific gene sequence is not limited thereto. In the present invention, TGF-β1 and TGF-β may be used interchangeably.

In the present invention, the term “oligonucleotide polymer including a repeating unit of an antisense against TGF-3” refers to an oligonucleotide polymer in which one or more antisenses against monomeric TGF-β are linked to form a polymer. The repeating unit is a unit constituting an oligonucleotide polymer, and refers to an antisense against TGF-β that is repeatedly linked plural times. The oligonucleotide polymer including a repeating unit of an antisense against TGF-β is obtained by forming a polymer from a monomeric antisense oligonucleotide (ASO), and thus it may be used interchangeably with a polymeric antisense oligonucleotide (polymeric ASO, pASO).

In the present invention, the oligonucleotide polymer including a repeating unit of an antisense against TGF-β may be a polymeric antisense oligonucleotide (pASO) against TGF-β, but is not limited thereto.

In addition, in the present invention, the antisense against TGF-β may specifically be an antisense against any one or more selected from the group consisting of TGF-β1, TGF-β2, and TGF-β3, and more specifically may be an antisense against TGF-β1, but is not limited thereto. In the present invention, TGF-β1 and TGF-β may be used interchangeably.

For the purposes of the present invention, the antisense against TGF-β1 may include the nucleotide sequence of SEQ ID NO: 1, but is not limited thereto.

The oligonucleotide polymer including a repeating unit of an antisense against TGF-β may be prepared by rolling circle amplification (RCA), but it may be prepared using any method known in the art without limitation as long as it can produce the oligonucleotide polymer.

In the present invention, the repeating unit of an antisense against TGF-β may repeat 1 to 1,000 times, specifically 10 to 500 times, and more specifically 50 to 300 times, but is not limited thereto.

SEQ ID NO: 1 described above may be a single-stranded nucleotide sequence complementary to the gene sequence encoding TGF-β1. The nucleotide sequence of SEQ ID NO: 1 can be obtained from a known database (i.e., NCBI GenBank). In an example, it may be of human origin, but is not limited thereto, and a sequence having the same activity as the nucleotide sequence above may be included without limitation. In addition, while it is defined that the oligonucleotide in the present invention includes the nucleotide sequence of SEQ ID NO: 1, it does not exclude addition of a meaningless sequence upstream or downstream of the nucleotide sequence of SEQ ID NO: 1, a mutation that may occur naturally, or a silent mutation thereof, and it is apparent to those skilled in the art that any oligonucleotide will correspond to the oligonucleotide of the present invention as long as it has an activity that is the same as or corresponding to that of the oligonucleotide including the nucleotide sequence of SEQ ID NO: 1. As a specific example, the oligonucleotide of the present invention may consist of the nucleotide sequence of SEQ ID NO: 1 or a nucleotide sequence having a homology or identity of at least 80%, 85%, 90%, 95%, 96%, 97%, 98%, or 99% or more to the nucleotide sequence of SEQ ID NO: 1. Additionally, it is apparent that any oligonucleotides having a nucleotide sequence, in which part of the nucleotide sequence is deleted, modified, substituted, or added, may also be included within the scope of the present invention as long as the nucleotide sequence has such a homology or identity of a nucleotide sequence to that of the above oligonucleotide and exhibits an effect corresponding to that of the above oligonucleotide.

That is, in the present invention, even when it is described as an “oligonucleotide consisting of a nucleotide sequence represented by a specific SEQ ID NO”, it is apparent that as long as it has an activity that is the same as or corresponding to that of the oligonucleotide consisting of the nucleotide sequence of the corresponding SEQ ID NO, the oligonucleotide consisting of a nucleotide sequence in which the sequence is partially deleted, modified, substituted, or added can also be used in the present invention. For example, it is apparent that an “oligonucleotide consisting of a nucleotide sequence of SEQ ID NO: 1” can belong to the “oligonucleotide consisting of a nucleotide sequence of SEQ ID NO: 1” of the present invention as long as it has an activity identical or corresponding thereto.

The biocompatible cationic peptide may be a dimeric β-defensin peptide in which two β-defensin peptides are polymerized, and the β-defensin peptide may be of human origin. That is, the biocompatible cationic peptide may be a dimeric human β-defensin peptide (DhBD), but is not limited thereto.

The dimeric β-defensin peptide may be prepared by oxidizing a cysteine residue in β-defensin in accordance with previously reported methods, but the preparation method is not limited thereto.

In the present invention, the “β-defensin” is a member of the defensin family. Defensins are proteins that are 2 to 6 kDa in size, are cationic, and contain three pairs of intramolecular disulfide bonds. Defensins show a bactericidal activity against many Gram-negative and Gram-positive bacteria, fungi, and enveloped viruses. Defensins originated from mammals are classified into alpha, beta, and theta categories based on the size and pattern of disulfide bonds. As far as known, β-defensins are present in all mammalian species. β-Defensins are produced by various cells. For example, in cattle, 13 β-defensins are present in neutrophils, but in other species, β-defensins are more often produced by epithelial cells that form the lining of various organs such as the epidermis, bronchi, and urogenital tract. β-Defensins are known as antimicrobial peptides associated with the resistance of the epithelial surface to microbial colonization.

There are various types of β-defensin, for example, β-defensin 1, β-defensin 103A, β-defensin 105A, β-defensin 105B, β-defensin 106, β-defensin 108B, β-defensin 109, (3-defensin 110, β-defensin 111, β-defensin 114, β-defensin 130, β-defensin 136, β-defensin 4, and β-defensin 23. Specifically, the β-defensin peptide may be any one selected from the group consisting of β-defensin 1, β-defensin 103A, β-defensin 105A, β-defensin 105B, β-defensin 106, β-defensin 108B, β-defensin 109, β-defensin 110, β-defensin 111, β-defensin 114, β-defensin 130, β-defensin 136, β-defensin 4, and β-defensin 23, and more specifically may be β-defensin 23 comprising the amino acid sequence of SEQ ID NO: 4, but is not limited thereto.

In the present invention, SEQ ID NO: 4 refers to an amino acid sequence having the activity of β-defensin 23. Specifically, SEQ ID NO: 4 described above is a protein sequence having the activity of β-defensin 23 which is encoded by a gene encoding β-defensin 23. The amino acid of SEQ ID NO: 4 can be obtained from a known database (i.e., NCBI GenBank). For example, it may be of human origin, but is not limited thereto, and a sequence having the same activity as the above amino acid may be included without limitation. In addition, while the protein having the activity of β-defensin 23 in the present invention is defined as a protein including the amino acid of SEQ ID NO: 4, it does not exclude addition of a meaningless sequence upstream or downstream of the amino acid sequence of SEQ ID NO: 4, a mutation that may occur naturally, or a silent mutation, and it is apparent to those skilled in the art that if it has an activity that is the same as or corresponding to that of a protein including the amino acid sequence of SEQ ID NO: 4, it will correspond to the β-defensin 23 of the present invention. Specifically, the β-defensin 23 of the present invention may be a protein consisting of the amino acid sequence of SEQ ID NO: 4 or of an amino acid sequence having a homology or identity of at least 80%, 85%, 90%, 95%, 96%, 97%, 98%, or 99% or more to the amino acid sequence of SEQ ID NO: 4. Additionally, it is apparent that any protein having an amino acid sequence, in which part of the amino acid sequence is deleted, modified, substituted, or added, may also be included within the scope of the protein of present invention to be mutated as long as the amino acid sequence has such a homology or identity of an amino acid sequence to that of the above protein and exhibits an effect corresponding to that of the above protein.

That is, in the present invention, even when it is described as a “protein or polypeptide having an amino acid sequence represented by a specific SEQ ID NO” or a “protein or polypeptide including an amino acid sequence represented by a specific SEQ ID NO”, it is apparent that a protein having an amino acid sequence in which the sequence is partially deleted, modified, substituted, or added can also be used in the present invention, as long as it has an activity that is the same as or corresponding to the polypeptide consisting of the amino acid sequence of the corresponding SEQ ID NO. For example, it is apparent that a “polypeptide consisting of an amino acid sequence of SEQ ID NO: 4” may belong to the “polypeptide consisting of an amino acid sequence of SEQ ID NO: 4” of the present invention as long as it has an activity identical or corresponding thereto.

As the pASO of the present invention is polyanionic, it can easily bind to multiple positively charged residues of DhBD to form a complex of pASO and DhBD in the form of nanoparticles. In addition, the complexation of pASO and DhBD increases a particle size so as to suit lung accumulation, protects ASO from nuclease degradation, and further enhances its absorption into fibrotic cells after arrival at the lung tissue. With this complexation of pASO and DhBD, the ASO successfully delivered to the fibrotic cells in the lungs of interest effectively downregulates the target gene TGF-β1, thereby suppressing pulmonary fibrosis in an animal model.

For the purposes of the present invention, the drug delivery system of the present invention, which is a complex consisting of pASO and DhBD, is absorbed into the lung cells by the action of DhBD to knock down TGF-β including the TGF-β1, 2, and 3 subfamilies by the action of pASO, thereby preventing or treating pulmonary fibrosis.

The fibrotic cell may be one or more of an endothelial cell or a fibroblast cell, but is not limited thereto.

In particular, after accumulation in the lung capillaries, microparticles need to be fragmented into smaller nanoparticles under physiological conditions to avoid pulmonary embolism and enhance endothelial cell uptake. The pASO/DhBD23 microparticles are formed by non-covalent interactions and composed of fully biocompatible materials, such as DNA and human-derived peptides, which are expected to be fragmented by shear stress or degraded by blood flow in the capillary. Indeed, the size of aggregates made by pASO/DhBD23 and albumin was decreased up to 500 nm after shear stress applied using pipetting. This is an advantage of pASO/DhBD23 to be used as a lung-targeted drug carrier over other types of microparticles that cannot be fragmented into nanoparticles under physiological conditions.

Another aspect of the present invention provides a pharmaceutical composition for the prevention or treatment of pulmonary fibrosis, comprising, as an active ingredient, a lung-specific drug delivery system consisting of oligonucleotide polymers, which include a repeating unit of an antisense against TGF-β, and biocompatible cationic peptides.

The terms as used herein are as described above.

The pharmaceutical composition of the present invention has a use in the “prevention” and/or “treatment” of pulmonary fibrosis. For the pharmaceutical composition for its use in the prevention, it is administered to a subject who has or is suspected of being at risk of developing the disease, disorder, or condition described herein. That is, it can be administered to a subject at risk of developing pulmonary fibrosis. For the pharmaceutical composition for its use in the treatment, it is administered to a subject, such as a patient already suffering from the disorder described herein, in an amount sufficient to treat or at least partially arrest the symptoms of the disease, disorder, or condition described herein. The amount effective for this use may vary depending on the severity and course of the disease, disorder, or condition, prior treatment, the individual's health status, responsiveness to the drug, and the determination of the physician or veterinarian.

It may further include a suitable carrier, excipient, or diluent commonly used in the preparation of the pharmaceutical composition of the present invention. The content of the active ingredient included in the composition is not particularly limited, but may be included at 0.0001 wt % to 10 wt %, preferably 0.001 wt % to 1 wt % based on the total weight of the composition.

The pharmaceutical composition may have any one formulation selected from the group consisting of tablets, pills, powders, granules, capsules, suspensions, liquid preparations for internal use, emulsions, syrups, sterilized aqueous solutions, non-aqueous solvents, freeze-dried agents, and suppositories, and may be of various oral or parenteral formulations. For formulation, the composition is prepared using commonly used diluents or excipients such as fillers, extenders, binders, wetting agents, disintegrants, and surfactants. Examples of the solid formulation for oral administration include tablets, pills, powders, granules, capsules, and the like, and these solid formulations are prepared by mixing one or more compounds with at least one excipient or more, such as starch, calcium carbonate, sucrose, lactose, gelatin, and the like. Additionally, in addition to simple excipients, lubricants such as magnesium stearate, talc, and the like are also used. Examples of the liquid formulation for oral administration include suspensions, liquid preparations for internal use, emulsions, syrups, and the like, and may include various excipients such as wetting agents, sweetening agents, fragrances, and preservatives in addition to water and liquid paraffin, which are commonly used simple diluents. Examples of formulation for parenteral administration include sterilized aqueous solutions, non-aqueous solutions, suspensions, emulsions, lyophilized formulations, and suppositories. For the non-aqueous solvent and suspension, propylene glycol, polyethylene glycol, vegetable oil such as olive oil, injectable ester such as ethyl oleate, and the like may be used. As a base for suppositories, witepsol, macrogol, tween 61, cacao butter, laurin butter, glycerogelatin, and the like may be used.

The composition of the present invention can be administered to a subject in a pharmaceutically effective amount.

In the present invention, the term “pharmaceutically effective amount” means an amount sufficient to treat a disease at a reasonable benefit/risk ratio applicable to medical treatment, and the level of effective dosage can be determined according to the type, severity, age, sex of the individual, type of disease, activity of the drug, sensitivity to the drug, time of administration, route of administration and rate of excretion, the duration of treatment, factors including the drugs used simultaneously, and other factors well known in the medical field. The composition of the present invention can be administered as an individual therapeutic agent or administered in combination with other therapeutic agents, and can be administered sequentially or simultaneously with a conventional therapeutic agent. In addition, the composition of the present invention can be administered alone or in combination. It is important to administer the pharmaceutical composition in the minimum amount that can exhibit the maximum effect without causing side effects, in consideration of all of the factors described above, which may be easily determined by those skilled in the art. The preferred dosage of the composition of the present invention may vary according to the condition and weight of the patient, the degree of the disease, the form of the drug, and the route and duration of administration, and the administration may be conducted once a day, or may also be conducted several times a day. The composition of the present invention can be administered to any subject that requires the prevention or treatment of pulmonary fibrosis, without particular limitation. The composition of the present invention can be administered by various conventional methods. For example, the composition of the present invention can be administered by oral or rectum administration, or by intravenous, intramuscular, subcutaneous, intrauterine dural, or cerebrovascular injection.

The pharmaceutical composition of the present invention can be administered to a subject who has developed and progressed or has a high likelihood of developing pulmonary fibrosis, thereby preventing the occurrence of pulmonary fibrosis or alleviating the degree of occurrence.

The drug delivery system according to the present invention may additionally include a drug for preventing or treating pulmonary fibrosis, and the drug may be used without limitation as long as it can be used for the prevention or treatment of pulmonary fibrosis.

Still another aspect of the present invention provides a method for preventing or treating pulmonary fibrosis, comprising a step of administering to a subject a composition comprising a lung-specific drug delivery system consisting of oligonucleotide polymers, which include a repeating unit of an antisense against TGF-β, and biocompatible cationic peptides.

The terms as used herein are as described above.

The antisense against TGF-β may be an antisense against TGF-β1, and the antisense against TGF-β1 may be one including a nucleotide sequence of SEQ ID NO: 1, but is not limited thereto. The repeating unit of the antisense against TGF-β may repeat 1 to 1,000 times.

The biocompatible cationic peptide may be a dimeric β-defensin peptide, and the β-defensin peptide may be of human origin, and specifically may be β-defensin 23, but is not limited thereto.

The drug delivery system may be absorbed into the cells of the lungs and knock down TGF-β to prevent or treat pulmonary fibrosis. The cells may be specifically at least one of endothelial cells or fibroblasts.

In the present invention, the term “subject” refers to all animals that have developed or may develop pulmonary fibrosis, and the pharmaceutical composition of the present invention can efficiently treat a subject by administering the pharmaceutical composition to the subject suspected of having pulmonary fibrosis.

In the present invention, the term “administration” means introducing the pharmaceutical composition of the present invention to a subject suspected of having pulmonary fibrosis by any suitable method, and as long as the route of administration can reach the target tissue, the composition of the present invention can be administered through various routes, either an oral or parenteral route.

The pharmaceutical composition of the present invention can be administered in a pharmaceutically effective amount.

In the present invention, the term “pharmaceutically effective amount” means an amount sufficient to treat a disease at a reasonable benefit/risk ratio applicable to medical treatment, and the level of effective dosage can be determined according to the type, severity, age, sex of the individual, type of disease, activity of the drug, sensitivity to the drug, time of administration, route of administration and rate of excretion, the duration of treatment, factors including the drugs used simultaneously, and other factors well known in the medical field. The composition of the present invention can be administered as an individual therapeutic agent or administered in combination with other therapeutic agents, and can be administered sequentially or simultaneously with a conventional therapeutic agent. In addition, the composition of the present invention can be administered alone or in combination. It is important to administer the pharmaceutical composition in the minimum amount that can exhibit the maximum effect without causing side effects, in consideration of all of the factors described above, which may be easily determined by those skilled in the art.

The pharmaceutical composition of the present invention can be administered to any subject that requires the prevention or treatment of pulmonary fibrosis, without particular limitation. For example, the composition of the present invention can be applied to any of non-human animals such as monkey, dog, cat, rabbit, guinea pig, rat, mice, cow, sheep, pig, goat, bird and fish, and so on, and the pharmaceutical composition can be administered parenterally, subcutaneously, intraperitoneally, intrapulmonarily, and intranasally. For topical treatment, if necessary, it can be administered by a suitable method including intralesional administration. The preferred dosage of the pharmaceutical composition of the present invention varies according to the condition and weight of the individual, the severity of the disease, the form of the drug, and the route and duration of administration, but may be appropriately selected by those skilled in the art. For example, the composition of the present invention can be administered by oral or rectal administration, or by intravenous, intramuscular, subcutaneous, intrauterine dural, or cerebrovascular injection, but is not limited thereto.

An appropriate total amount of administration per 1 day of the pharmaceutical composition of the present invention can be determined by a physician within the range of correct medical determination, and is generally 0.001 mg/kg to 1,000 mg/kg, preferably 0.05 mg/kg to 200 mg/kg, more preferably 0.1 mg/kg to 100 mg/kg once a day, or can be administered in divided doses multiple times daily.

### Advantageous Effects

The drug delivery system according to the present invention can be specifically accumulated in the lungs and absorbed into lung fibrotic cells to knock down TGF-β, thereby preventing or treating pulmonary fibrosis.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Schematic illustration for preparation of the pASO/DhBD23 complex and delivery of the complex to the lungs to treat pulmonary fibrosis by downregulation of TGF-β.

FIG. 2. The sequences used for rolling circle amplification and dimerization of hBD23. The primer binding sequence is indicated in blue. The complementary sequence to ASOs (sense) is indicated in orange. The linker sequence is indicated in gray.

FIG. 3. Preparation of the circular template and DhBD23. (a) Confirmation of production of the circular template with 8% denaturing PAGE gel. (b) Confirmation of dimerization of hBD23 with 17% SDS-PAGE gel.

FIG. 4. Characterization of pASO and pASO/DhBD23. (a) Agarose gel electrophoresis (0.5%) of pASO complexed with DhBD23 (pASO:DhBD23) with various ammonium/phosphate (N/P) ratios. (b) Dynamic light scattering analysis of pASO and pASO/DhBD23. (c) Zeta potential measurement of pASO and pASO/DhBD23. Scanning electron microscopy images of (d) pASO and (e) pASO/DhBD23 (scale bar=500 nm). (f) Quantitative serum stability estimated using electrophoretic analysis of (g) pASO and (h) pASO/DhBD23 (mean±SEM, n=3; ***P<0.001 by unpaired t-test). The pASO and pASO/DhBD23 were incubated in 50% mouse serum for pre-determined time points and analyzed in 2% agarose gel. S denotes serum only.

FIG. 5. Size change of the pASO/DhBD23/BSA complex under shear stress measured by DLS (mean±SEM, n=5; ***P<0.001 between (−) and (+) shear stress, paired t-test).

FIG. 6. Evaluation of in vitro activity of pASO and pASO/DhBD23. qRT-PCR analysis of TGF-β mRNA level in the MLg cells (mean±SEM, n=5; ***P<0.001 vs. pASO and pASO/DhBD23; ##P<0.01 between pASO and pASO/DhBD23).

FIG. 7. Cellular uptake property of pASO and pASO/DhBD23. Flow cytometry analysis of cellular uptake of pASO and pASO/DhBD23 in (a) bEnd.3 cells and (b) MLg cells (mean±SEM, n=3; *P<0.05 and ***P<0.001 vs. PBS; #P<0.05 and ###P<0.001 vs. pASO/DhBD23). (c) Fluorescence microscopic images of (c) bEnd.3 cells and (d) MLg cells treated with pASO and pASO/DhBD23 (magnification: ×400; scale bar: 20 μm). (e) Fluorescence microscopic images of MLg cells treated with pASO/DhBD23 and LysoTracker. (magnification: ×400; scale bar: 20 μm).

FIG. 8. Cellular uptake mechanism of the pASO/DhBD23 complex into bEnd.3 (mean±SEM, n=3; **P<0.01 vs. No Inhibitor).

FIG. 9. Biodistribution study in the pulmonary fibrosis mouse model. (a) Time-dependent in vivo biodistribution images of mice after intravenous injection of pASO and pASO/DhBD23. (b) Ex vivo images of major organs excised from mice 7 h post-injection. (c) Fluorescence intensity measured in ex vivo imaging (mean±SEM, n=3). (d) Tissue section images of the lung and the liver stained with DAPI (magnification: ×200; scale bar: 50 μm).

FIG. 10. Time-course monitoring of in vivo fluorescence intensity of pASO and pASO/DhBD23 at the lung.

FIG. 11. Ex vivo images of major organs from pulmonary fibrosis mice 7 h post-injection (n=3).

FIG. 12. Size change of pASO and pASO/DhBD23 upon binding with serum protein measured by DLS.

FIG. 13. Ex vivo images of major organs from healthy mice 7 h post-injection (n=3).

FIG. 14. In vivo TGF-β1 gene knockdown using RCA/DhBD23 complexes.

### DETAILED DESCRIPTION

Hereinafter, the present invention will be described in more detail by way of examples. However, it will be apparent to those of ordinary skill in the art to which the present invention pertains that these examples are for illustrative purposes of the present invention, and the scope of the present invention is not limited by these examples.

### Example 1. Formulation of pASO/DhBD23 Complex

1-1. Circularization and RCA of Template DNA

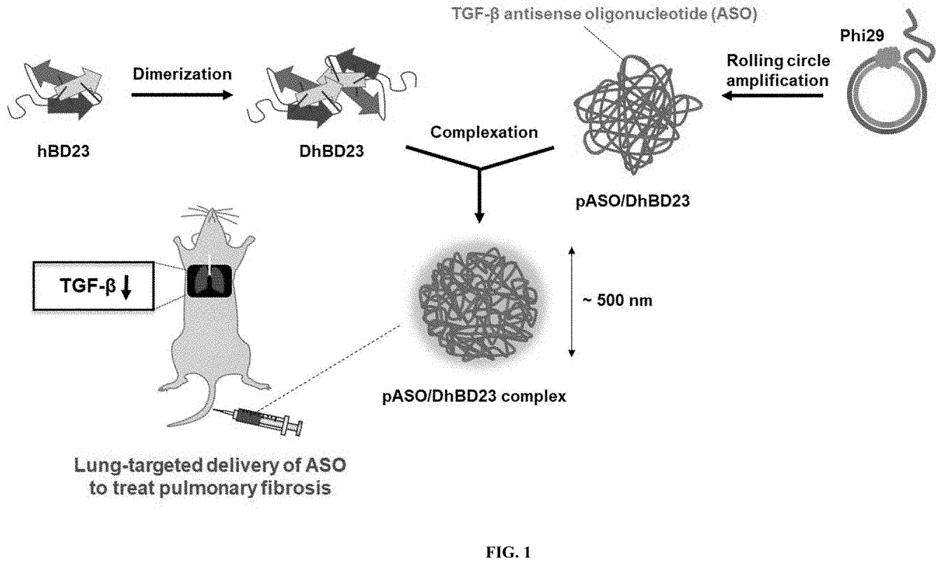

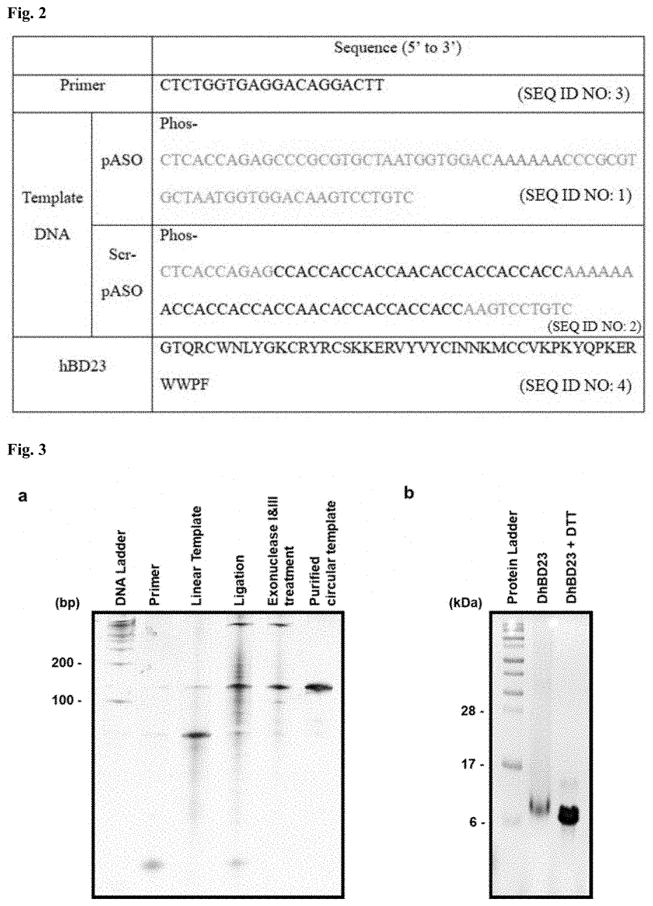

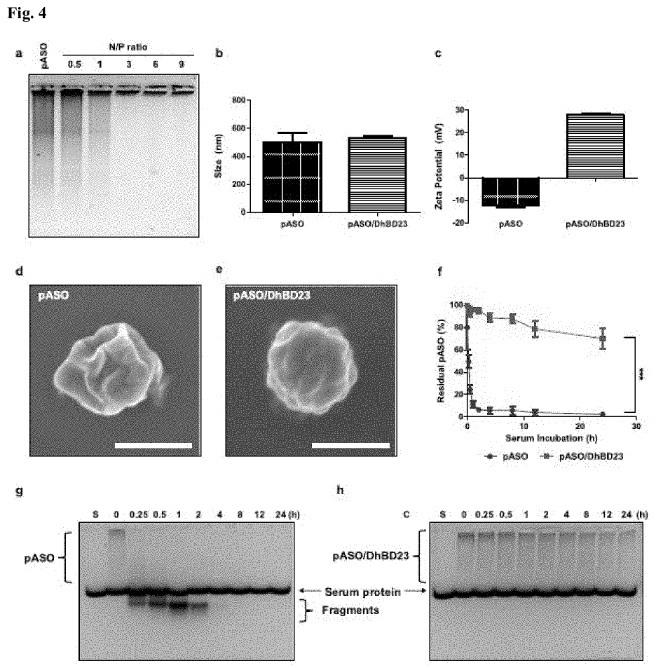

For the RCA reaction, a linear template oligonucleotide containing two copies of antisense oligonucleotides (ASO) for TGF-β1 and a linear template oligonucleotide (Phos-CTCACCAGAGCCCGCGTGCTAATGGTGGACAAAAAACCCGCGTGCTAATGGTGGA CAAGTCCTGTC, SEQ ID NO: 1, FIG. 2) with phosphate bonded to the terminal was prepared and circularized by a T4 ligase reaction. 15 μM pASO primer (Bioneer, Korea), 10 μM template DNA, T4 ligase, and ligation buffer were mixed and incubated at 16° C. for overnight. Uncircularized oligonucleotides were removed by enzymatic reaction at 37° C. for 2 hours using exonucleases I and III. The circularized template DNA was separated on 8% denaturing polyacrylamide gel electrophoresis (PAGE) and purified using an ethanol precipitation method. The purified circular DNA template was quantified at a wavelength of 280 nm using a UV spectrophotometer, and identified by 8% denaturing PAGE at 100 V for 80 minutes (FIG. 3a). As illustrated in FIG. 1, the RCA reaction on the circular template yielded pASO as the high-molecular-weight product, demonstrated using agarose gel electrophoresis (FIG. 4a, leftmost lane), and then the RCA reaction was performed using phi29 polymerase.

50 nM RCA primer (SEQ ID NO: 3), 50 nM circular template DNA, 0.2 mM dNTP, phi29 buffer, and phi29 polymerase were mixed and incubated at 30° C. for 1 hour. The reaction was terminated by incubating at 65° C. for 10 minutes. The reaction mixture was purified by dialysis at 4° C. for 3 days using a 100 K Amicon membrane filter (Merck Millipore, Burlington, Mass., USA). The purified RCA product was quantified with UV spectrophotometry at a wavelength of 260 nm.

The RCA reaction on the circular template yielded pASO ([CTCACCAGAGCCCGCGTGCTAATGGTGGACAAAAAACCCGCGTGCTAATGGTGG ACAAGTCCTGTC]ₙ, n is 100-200) as the high-molecular-weight product, demonstrated using agarose gel electrophoresis.

1-2. Dimerization of hBD23 Peptides

As biocompatible cationic peptides to be complexed with pASO, dimeric human β-defensin peptides (DhBD) were used. Among various human β-defensins (hBDs), monomeric β-defensin 23 (hBD23) (SEQ ID NO: 4, FIG. 2) was selected since it is composed of a relatively short sequence that can be chemically synthesized. To stabilize the morphology of hBD23, a cysteine residue in hBD23 was dimerized using an oxidation reaction. 50 μM hBD23, 5 mM cysteine, 500 μM cysteine, 2 mM ethylenediaminetetraacetic acid (EDTA), 1 M guanidine chloride, and 0.1 M ammonium acetate were mixed and incubated at 4° C. while stirring overnight. By centrifugation at 1,500 g for 1 hour using a 3K Amicon tube, the reaction mixture was purified, and the buffer was exchanged with distilled water. Purified dimerized-hBD23 (DhBD23) was quantified with UV spectrophotometry at a wavelength of 280 nm, and peptides were verified on 17% sodium dodecyl sulfate-polyacrylamide gel electrophoresis (SDS-PAGE) performed at 200 V for 40 minutes (FIG. 3b) followed by Coomassie blue staining.

1-3. Complexation of pASO and DhBD23

Various N/P ratios between pASO and DhBD23 (the number of the ammonium groups in DhBD23/the number of the phosphate groups in pASO) were explored to determine the ratio at which all of the pASO was fully complexed with DhBD23. Various N/P ratios of pASO and DhBD23 (N/P ratio=0.5:1, 1:1, 3:1, 6:1, and 9:1) were mixed and incubated at room temperature for 1 hour while shaking. The complexation between pASO and DhBD23 was confirmed by agarose gel electrophoresis performed at 120 V for 40 minutes (FIG. 4a, lanes 2 to 6 from the left).

1-4. Measurement of Particle Size and Surface Charge of pASO/DhBD23 Complex

The diameter and surface charge (N/P ratio=3:1) of the pASO/DhBD23 complex were measured using a particle analyzer (Malvern instrument, Worcestershire, United Kingdom). To visualize the formulation, pASO and pASO/DhBD23 were dried on a silicon wafer and coated with Au. A high-resolution image of the sample was obtained at an acceleration voltage of 3 kV using Nova-SEM (Nova Nano SEM 200).

As a result, the size (504 nm) of pASO measured by dynamic light scattering (DLS) was increased to 541 nm after complexation with DhBD23 (FIG. 4b). The sizes of the complexes estimated by the scanning electron microscope were found to be slightly larger than those determined by DLS, which were approximately 560 nm for pASO and 600 nm for pASO/DhBD23 (FIGS. 4d and 4e).

Meanwhile, after accumulation in the lung capillary, microparticles need to be fragmented into smaller nanoparticles under physiological conditions to avoid pulmonary embolism and enhance endothelial cell uptake. The pASO/DhBD23 microparticles are formed by non-covalent interactions and composed of fully biocompatible materials, such as DNA and human-derived peptides, which are expected to be fragmented by shear stress or degraded by blood flow in the capillaries. Indeed, the size of aggregates made by pASO/DhBD23 and albumin was decreased up to 500 nm after shear stress applied using pipetting (FIG. 5). This is an advantage of pASO/DhBD23 to be used as a lung-targeted drug carrier over other types of microparticles that cannot be fragmented into nanoparticles under physiological conditions.

The complexation led to the morphological change from a distinctive flower-like shape of pASO to a particle shape with less concave faces in the complex in the SEM images. The negative zeta potential value of pASO (−12.0 mV) changed to +27.8 mV through the complexation with cationic DhBD23 (FIG. 4c).

1-5. In Vitro Activity of the pASO/DhBD23 Complex

Following the preparation and characterization of the pASO/DhBD23 complex, an in vitro activity of the complex was investigated. After the mouse lung fibroblasts (MLg) were treated with the complex, the level of TGF-β mRNA was estimated by quantitative reverse-transcriptase PCR (qRT-PCR).

200 nM of formulations (PBS, scrambled sequence of pASO (ScrpASO, SEQ ID NO: 2, FIG. 2), DhBD23, Scr-pASO/DhBD23, pASO, and pASO/DhBD23) in serum-free media were treated to MLg cells at 37° C. for 24 hours to examine the efficacy of the pASO/DhBD23 complex. Then, total RNA was isolated from the sample using a TRIzol® reagent (Qiazen, CA, USA). RNA purity and concentration were measured with a Nanodrop (Thermo Fisher Scientific, MA, USA). RNA was reverse-transcribed using a ReverTra Ace® qPCR RT Kit (TOYOBO, Osaka, Japan) according to the manufacturer's protocol. The mRNA expression was assessed by real-time PCR using SensiFAST sybr Hi-Rox Mix (Bioline USA Inc., MA, USA) with the CFX96 Touch™ Real-Time PCR Detection System (Bio-Rad, CA, USA). The 2-ΔΔCt method was used to analyze the relative changes in gene expression based on real-time quantitative PCR. GAPDH was used as an internal control gene. Primer sequences for qRT-PCR were as follows.

TGF-beta_Forward:
(SEQ ID NO: 5)
CGA AGC GGA CTA CTA TGC TAA AGA G
TGF-beta_Reverse:
(SEQ ID NO: 6)
TGG TTT TCT CAT AGA TGG CGT TG
GAPDH_Forward:
(SEQ ID NO: 7)
TGC ACC ACC AAC TGC TTA G
GAPDH_Reverse:
(SEQ ID NO: 8)
GGA TGA GGG ATG ATG TTC

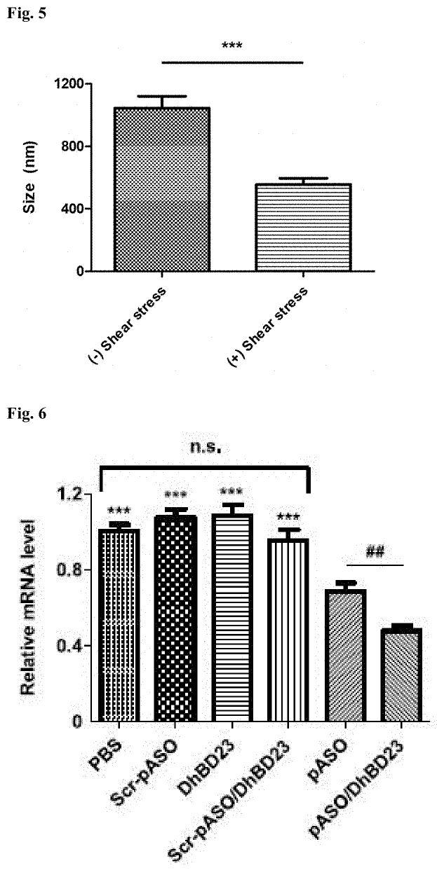

The target gene level was decreased by 31% upon treatment with pASO and 52% upon treatment with pASO/DhBD23 (FIG. 6). Treatment with DhBD23 did not significantly affect the target gene level. Moreover, the downregulation activity by pASO/DhBD23 appeared to be specific for the target sequence, as treatment with pASO composed of scrambled sequences such as Scr-pASO and ScrpASO/DhBD23 failed to induce the downregulation activity at the target gene.

### Example 2. Assay of Serum Stability of pASO/DhBD23 Complex

It is necessary to examine whether the serum stability of pASO is suitable for in vivo applications. To this end, 50% mouse serum was added to pASO and pASO/DhBD23 (2 μM), and the mixture was incubated at 37° C. for predetermined time periods (0, 0.25, 0.5, 1, 2, 4, 8, 12, and 24 hours). The incubated mixture was analyzed on 2% agarose gel electrophoresis at 120 V for 1 hour, stained with SYBR gold, and visualized using iBright™ FL1000 (Invitrogen, USA). Band intensity was quantified with ImageJ.

As a result, in the 50% mouse serum solution, 90% of pASO was degraded within 1 hour, whereas only 5% of pASO in the complex with DhBD23 was degraded (FIGS. 4f, 4g, and 4h). The pASO/DhBD23 complex maintained 70% of its integrity even after 24 hours. The significantly improved serum stability of pASO in the complex indicated that DhBD23 could protect the polynucleotide against serum nucleases.

### Example 3. Cell Uptake Analysis of pASO/DhBD23 Complex

Since the pASO/DhBD23 complex needs to penetrate into the endothelial cells of capillaries and reach fibrotic cells for high accumulation in the lung fibrous tissue, the degree of absorption of fibroblast cells was analyzed.

MLg cells (model lung fibroblast cells; mouse lung fibroblast cell line) and bEnd.3 cells (mouse brain endothelial cell line) were maintained in DMEM media (Welgene, Gyeongsan, Korea) supplemented with 10% fetal bovine serum (FBS, Welgene, Gyeongsan, Korea), 100 IU/mL penicillin, and 100 μg/mL streptomycin at 37° C. under a humidified atmosphere of 95% air and 5% CO₂.

pASO/DhBD23 was fluorescently labeled (Cy5-pASO/DhBD23) by exchanging 5% of dTTP with Cy5-dUTP (cyanine 5-6-propargylamino-2′-deoxyuridine-5′-triphosphate) when the RCA reaction was performed. The MLg cells and bEnd.3 cells (1×10⁵ cells/mL) were treated with 200 nM Cy5-pASO/DhBD23 complexes in serum-free media and incubated at 37° C. for 4 hours. The cells were washed two times with ice-cold PBS. Average fluorescence intensity was measured using flow cytometry (Merk KGaA, Darmstadt, Germany).

For fluorescence microscopy analysis, the MLg cells treated with Cy5-pASO/DhBD23 were fixed with 4% formaldehyde solution for 5 minutes. After washing, the nuclei were stained with DAPI (4′,6-diamidino-2-phenylindole). Subsequently, the fluorescence signal was visualized at a magnification of about ×200 using a fluorescence microscope.

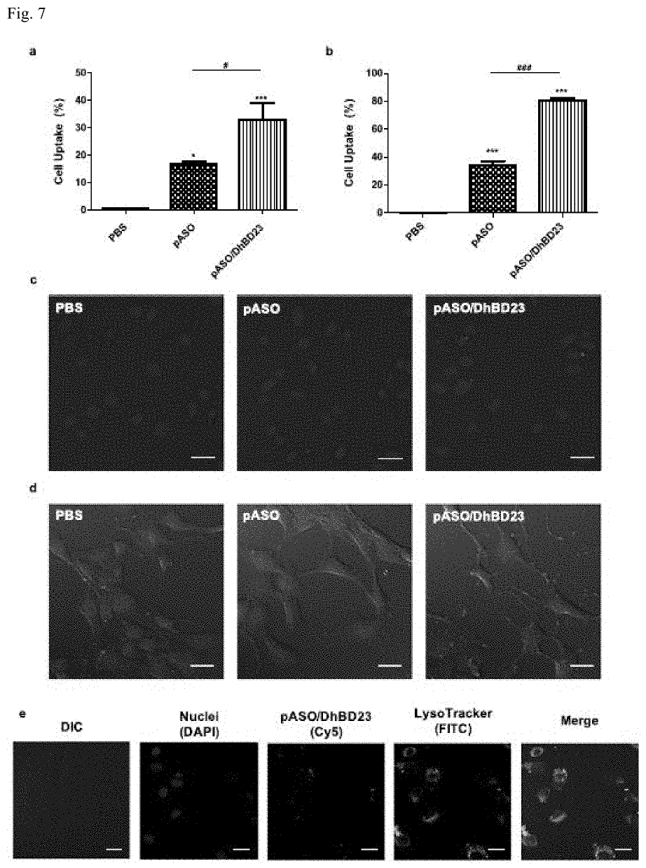

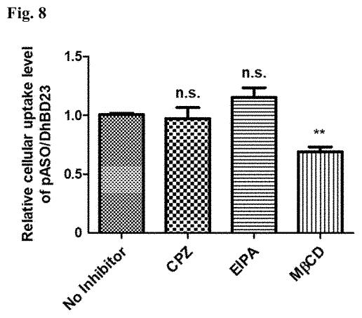

As a result, the flow cytometric analysis of the cells treated with pASO/DhBD23 demonstrated significantly enhanced cellular uptake compared to pASO (FIG. 7a). The endothelial cell uptake of pASO/DhBD23 was mediated by caveolae-dependent endocytosis (FIG. 8). Caveolae is abundantly expressed in lung endothelia and regulates transport of materials across the endothelial layer, which suggests that the complex could penetrate the lung endothelium via a caveolae-dependent mechanism.

In addition, pASO/DhBD23 showed remarkably improved cell uptake characteristics compared to pASO (FIG. 7b), and was more efficiently delivered to MLg cells. This indicates that pASO/DhBD3 can be more efficiently delivered to target fibroblasts after penetration of the capillary endothelial cell barrier than pASO.

Likewise, the high cellular uptake efficiency of the pASO/DhBD23 complex was observed in fluorescence microscopy images, while the uptake level of pASO was not different from that of the control group (FIGS. 7c and 7d). The intense red aggregates in cell images (FIGS. 7c and 7d) appear to be the complex particles concentrated in endosomes/lysosomes during endocytosis, as their locations are superimposed with the lysosomes visualized with LysoTracker (FIG. 7e). The complex particles could be diffused after cytosolic release.

These results suggest that the high lung accumulation of pASO/DhBD23 is due to the efficient penetration of the endothelial barrier and enhanced intracellular uptake into lung fibrotic cells after arriving at the lung capillary.

### Example 4. Assay of pASO/DhBD23 Complex for Lung-Specific Accumulation and TGF-β Knockdown Effect in Bleomycin-Induced Pulmonary Fibrosis (PF) Mouse Model

4-1. Bleomycin-Induced PF Mouse Model Preparation

All animal experiments were approved by the Institutional Animal Care and Use Committee (IACUC) of the Korea Institute of Science and Technology (KIST). The protocol of animal experiments was accorded with the KIST guidelines and regulations. Nine-week-old male C57BL6 mice (Orient Bio, Seongnam, Korea) were intratracheally injected with 50 μL of 3 mg/mL bleomycin (Sigma Aldrich).

The bleomycin-induced lung fibrosis mouse model presents a fibrotic histologic pattern and fibrosis-related biological factors in the lung (e.g., increased production of collagen and upregulation of TGF-β).

In vivo and ex vivo fluorescence imaging of the mouse model was performed for 3 days after injection of bleomycin, and molecular analysis and immunohistochemical experiments were performed 1 day after injection of bleomycin.

4-2. In Vivo and Ex Vivo Fluorescence Imaging

At 24 hours of post-injection of bleomycin, PF mice in each group were intravenously administered with PBS, Cy5-pASO, and Cy5-pASO/DhBD23, respectively (200 pmol RCA product/mouse). In vivo fluorescence imaging was performed at 1, 2, 3, 4, 5, 6, 7, 8, and 24 hours post-injection of PBS Cy5-pASO and Cy5-pASO/DhBD23 using the IVIS imaging system (PerkinElmer, Waltham, Mass., USA).

For ex vivo fluorescence imaging, PF mice in each group (n=3) intravenously injected with each sample (200 pmol RCA product/mouse) were sacrificed at 7 hours post-injection of PBS, Cy5-pASO, and Cy5-pASO/DhBD23. Fluorescence intensities in liver, lung, spleen, kidney, and heart were measured using the IVIS imaging system. Each organ immersed in an optimal cutting temperature (OCT) compound (Sakura Finetek USA, Inc., Torrance, Calif., USA) was frozen at −80° C. for frozen-sectioned fluorescence imaging.

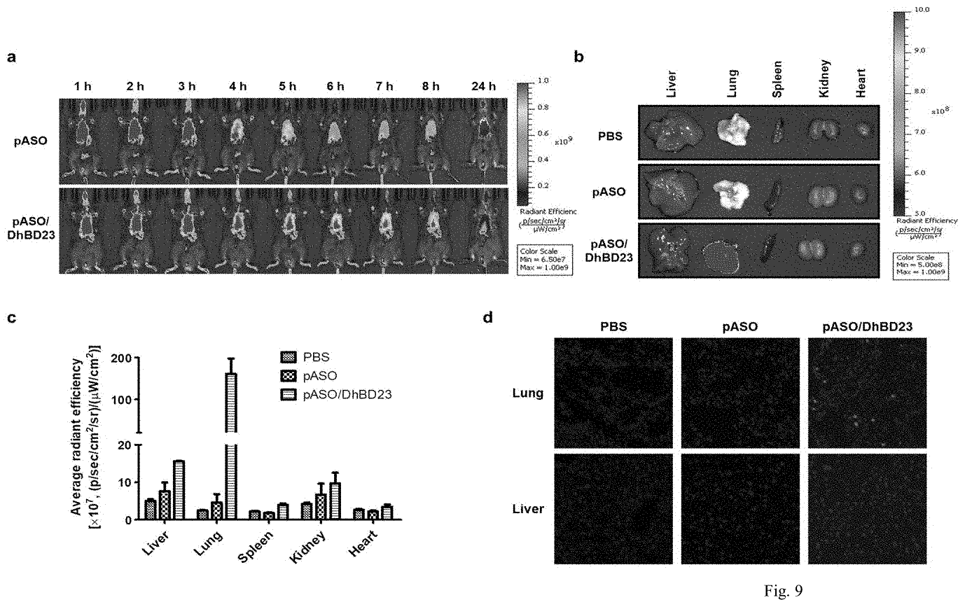

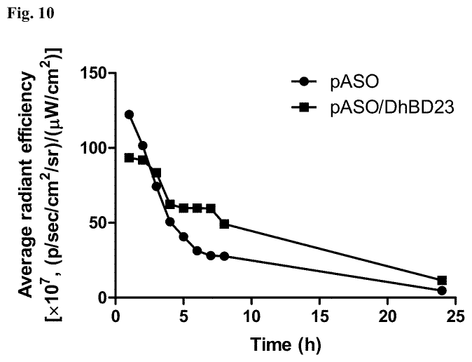

As a result, upon intravenous injection, pASO/DhBD23 was rapidly distributed in the lungs and then slowly removed over time (FIG. 9). Even at 24 hours post-injection, a considerable amount of pASO/DhBD23 remained in the lungs. In contrast, despite the similar initial lung distribution level, pASO was significantly cleared from the lungs after 24 hours (FIG. 10).

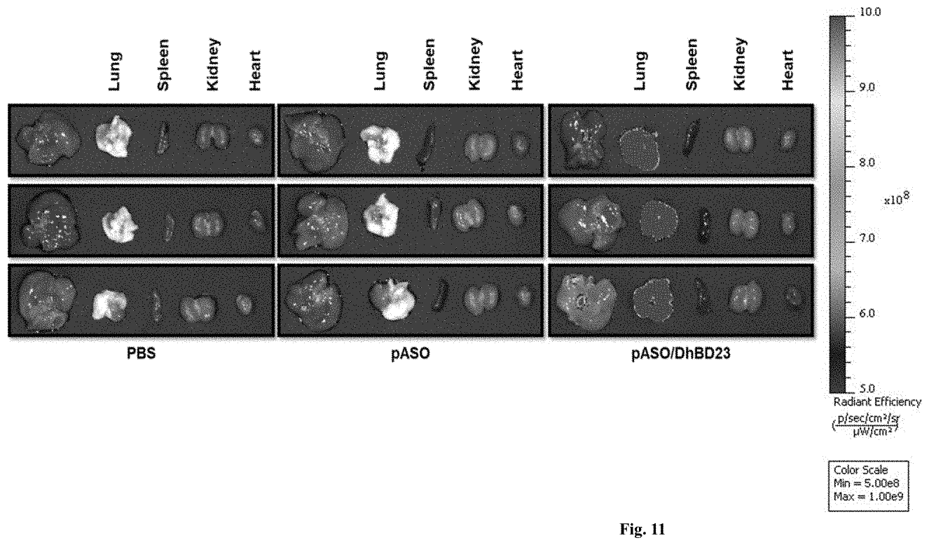

In ex vivo imaging of major organs at 7 hours post-injection, only pASO/DhBD23 showed distinguishably lung-specific accumulation (FIG. 9b). To quantitatively estimate the lung specificity of pASO/DhBD23, the averaged fluorescence intensity of each organ was measured (FIGS. 9c and 11). The intensity of pASO/DhBD23 in the lungs was approximately 10-fold higher than that in the liver, and the organ showed the second-highest accumulation level, which clearly indicated the high lung specificity of pASO/DhBD23. Additionally, the lung-specific accumulation was confirmed in the tissue section images obtained from the pASO/DhBD23-treated mice (FIG. 9d).

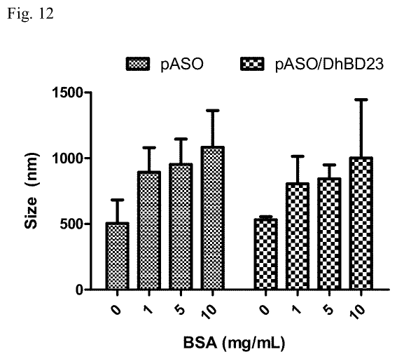

It is well known that lung accumulation can be achieved using micrometer-sized particles captured in the pulmonary capillary. When the hydrodynamic size was measured by DLS in the presence of albumin, the most abundant protein in serum, the size of the pASO/DhBD23 complex was increased to approximately 1 μm owing to binding with the serum protein. This suggests that the complex size was suitably increased for lung accumulation by adsorption of serum proteins after intravenous injection (FIG. 12). Similarly, the size of pASO was increased with the addition of albumin, which may contribute to the initial lung accumulation of the naked polynucleotide. However, despite enhancing transient lung accumulation, the size effect alone was insufficient to maintain a sustainably high lung distribution level.

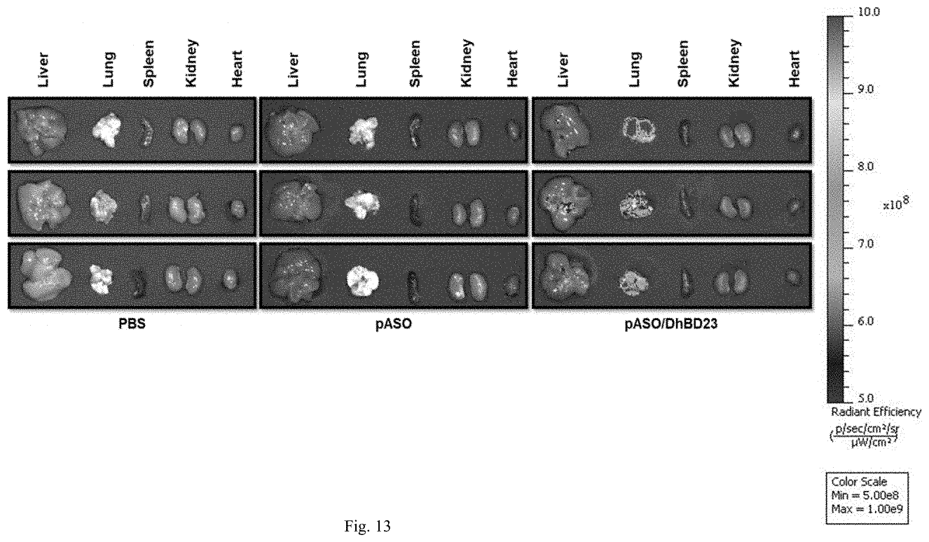

While the lung-targeting ability of the complex was not limited to the lung fibrosis model, as similar biodistribution was observed in healthy mice (FIG. 13), the accumulation level in healthy mice was lower than that in fibrosis mice. Once the complex particles are located in the lung capillaries based on the size effect, the shear stress due to blood flow can fragment the microparticles formed with nucleic acids and peptides into smaller nanoparticles which can be taken up into endothelial cells by endocytosis. Some of endocytosed nanoparticles will reach the interstitium via transcellular delivery. This is the size-dependent mechanism commonly shared in both healthy and fibrosis mice. In addition, according to previous studies, fibrosis can cause damage to the endothelium resulting in enhanced permeability of nanoparticles from capillaries to the interstitium where fibrotic cells are located, which explains the higher lung accumulation level of the complex in fibrosis mice compared with healthy mice.

4-3. Frozen-Sectioned Fluorescence Imaging

Frozen tissues were sectioned using a cryostat with a thickness of 10 μm. The nucleus in the tissue section was stained with DAPI mounting solutions (Abcam, Cambridge, United Kingdom). Fluorescence signals in the tissue sections were visualized using a fluorescence microscope at a magnification of ×200.

As a result, as in the ex vivo imaging results, only pASO/DhBD23 showed distinguishable lung-specific accumulation (FIG. 9d).

4-4. Western Blot

After observing the lung-specific delivery of pASO/DhBD23, in order to evaluate whether the delivered pASO can downregulate the target gene, 1 day after bleomycin injection, the PF mice in each group (n=3) were then intravenously administered with PBS, pASO, and pASO/DhBD23 (200 pmol RCA product/mouse). After 48 hours, the mice were sacrificed and the lungs excised and lysed, and it was evaluated by western blot whether there was knockdown of the TGF-β1 gene in the lung tissue.

The lung tissue was lysed in RIPA containing protease inhibitors at 4° C. for overnight. The cell lysis solution was centrifuged at 12,000 rpm at 4° C. for 20 minutes to obtain a cell pellet. The supernatant containing a protein was quantified by Bradford assay. 20 μg of the protein extract was transferred to 4% to 12% SDS-PAGE, separated, and then transferred to a PVDF membrane (100 minutes, 350 mA, 100 V), followed by blocking in the TBST buffer containing 5% BSA (w/v) at room temperature for 1 hour. The membrane was incubated at 4° C. overnight with a primary antibody, a rabbit anti-TGF-β1 antibody, and a rabbit anti-β-actin antibody (1:1,000). In the next day, the membrane was incubated for 2 hours in goat anti-rabbit IgG (1:3000) bonded with HRP as a secondary antibody. Finally, the target protein was visualized with a kit (SuperSignal West Pico Kit, Thermo Scientific) by Ibright (in vitrogen). The amount of expressed TGF-β1 was quantified using Image J.

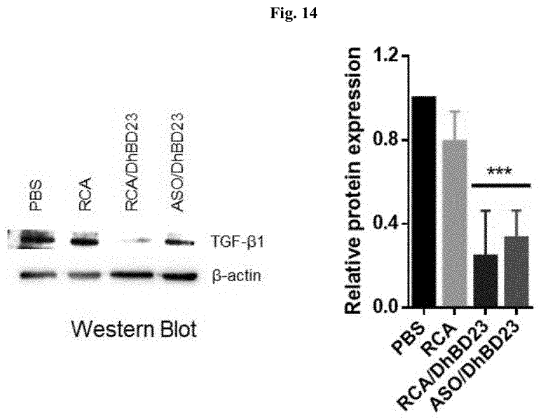

As a result, it was confirmed that pASO/DhBD23 successfully knocked down TGF-β1 under an in vivo environment as shown in FIG. 14, and showed superior knockdown efficiency than the control group (pASO and ASO/DhBD23).

From the above description, those skilled in the art to which the present invention pertains will be able to understand that the present invention can be embodied into different and more detailed modes, without departing from the technical spirit or essential features thereof. In this regard, it will be understood that the embodiments described above are only illustrative, and should not be construed as limiting. The scope of the present invention should be construed that all changes or modifications derived from the meaning and scope of the claims to be described below and equivalent concepts thereof, rather than the above detailed description are included in the scope of the present invention.

1. A lung-specific drug delivery system consisting of oligonucleotide polymers, which comprise a repeating unit of an antisense against TGF-β, and biocompatible cationic peptides.

2. The lung-specific drug delivery system of claim 1, wherein the antisense against TGF-β is an antisense against TGF-β1.

3. The lung-specific drug delivery system of claim 2, wherein the antisense against TGF-β1 comprises a nucleotide sequence of SEQ ID NO: 1.

4. The lung-specific drug delivery system of claim 1, wherein the repeating unit of the antisense against TGF-β repeats 1 to 1,000 times.

5. The lung-specific drug delivery system of claim 1, wherein the biocompatible cationic peptides are dimeric β-defensin peptides.

6. The lung-specific drug delivery system of claim 5, wherein the β-defensin peptides are of human origin.

7. The lung-specific drug delivery system of claim 6, wherein the β-defensin peptides are β-defensin 23.

8. The lung-specific drug delivery system of claim 1, wherein the drug delivery system is absorbed into cells of a lung and knocks down TGF-β to prevent or treat pulmonary fibrosis.

9. The lung-specific drug delivery system of claim 8, wherein the cells are at least one of endothelial cells or fibroblast cells.

10. A pharmaceutical composition comprising, as an active ingredient, a lung-specific drug delivery system consisting of oligonucleotide polymers, which comprise a repeating unit of an antisense against TGF-β, and biocompatible cationic peptides.

11. A method for preventing or treating pulmonary fibrosis, comprising a step of administering to a subject a composition comprising a lung-specific drug delivery system consisting of oligonucleotide polymers, which comprise a repeating unit of an antisense against TGF-β, and biocompatible cationic peptides.

12. The method of claim 11, wherein the antisense against TGF-β is an antisense against TGF-β1.

13. The method of claim 12, wherein the antisense against TGF-β1 comprises a nucleotide sequence of SEQ ID NO: 1.

14. The method of claim 11, wherein the repeating unit of the antisense against TGF-β repeats 1 to 1,000 times.

15. The method of claim 11, wherein the biocompatible cationic peptides are dimeric β-defensin peptides.

16. The method of claim 15, wherein the β-defensin peptides are of human origin.

17. The method of claim 16, wherein the β-defensin peptides are β-defensin 23.

18. The method of claim 11, wherein the drug delivery system is absorbed into cells of a lung and knocks down TGF-β to prevent or treat pulmonary fibrosis.

19. The method of claim 18, wherein the cells are at least one of endothelial cells or fibroblast cells.
